(19) United States
(12) Patent Application Publication
(10) Pub. No.: US 2023/0142104 A1
(43) Pub. Date: May 11, 2023

(54) METHOD AND DEVICE FOR GENERATING A CAMERA IMAGE OF A WELDING SEAM FOR AN IMAGE PROCESSING-SUPPORTED LASER TRANSMISSION WELDING METHOD

(71) Applicant: JENOPTIK Optical Systems GmbH, Jena (DE)

(72) Inventors: Stefan FRANZ, Jena (DE); Jan WERSCHNIK, Jena (DE); Steffen REINL, Eichenbuehl (DE); Sebastian TASCHNER, Apolda (DE); Benedikt BRANDAU, Jena (DE)

(21) Appl. No.: 17/906,957

(22) PCT Filed: Mar. 22, 2021
(86) PCT No.: PCT/DE2021/100285

§ 371 Date: Sep. 21, 2022

(30) Foreign Application Priority Data
Mar. 25, 2020 (DE) 10 2020 108 289.4

(51) Int. Cl. B23K 26/03; B29C 65/16; B23K 26/24; B23K 31/12

(52) U.S. Cl. CPC B23K 26/032; B29C 65/1635; B23K 26/24; B23K 31/125

(57) ABSTRACT

A method for generating a camera image from which a welding contour can be derived along which an assembly is to be welded in an image processing-supported laser transmission welding method. The transparent component of the assembly is illuminated from a side facing away from the camera. The invention also relates to a device which is suitable to carry out the method and wherein an illumination device has at least one light source, the light source being arranged in a workpiece holder in which the assembly to be welded is received in a receiving area and being directed into the receiving area.

### PRIORITY CLAIM

The present application is a National Phase entry of PCT Application No. PCT/DE2021/100285, filed Mar. 22, 2021, which claims priority from German Patent Application No. 10 2020 108 289.4, filed Mar. 25, 2020, the disclosures of which are hereby incorporated by reference herein in their entirety.

### BACKGROUND OF THE INVENTION

In image processing-supported manufacturing processes, control signals for carrying out the manufacturing process are derived from camera images. This type of control is of interest when the component tolerances are so large that a fixed movement regime of the tool does not lead to a desired processing result.

The camera image can be the image of a component to be processed or of details of the component, from which the position, orientation and shape of the component or of the detail of the component to be processed can be derived. The detection of these properties technically requires contrast generation in the camera image, i.e. the contour of the component or the detail of interest must stand out clearly from the background in the camera image. Conventionally, for the illumination of the component or the detail of interest, illumination systems are used which illuminate the object plane of the camera with an illumination beam path coaxial or inclined to the imaging beam path of the camera.

In laser transmission welding, a special feature is that the contour to be processed (welding contour) is formed by a contact area via which a component that is transparent to the laser radiation and a component that absorbs the laser radiation are in contact. This means that, starting from a light source of a conventionally arranged illumination system, the welding contour to be captured by the camera is located in the illumination beam path behind the component that is transparent to the laser radiation. This leads at least to a reduction in contrast of the image, particularly if the surface of the transparent component is uneven and thus has a scattering effect.

In order to be able to control welding along the welding contour with the aid of image processing, the welding contour must be clearly recognizable in the camera image. This is only possible if the two components to be welded are in contact only along the welding contour. This is the case when the welding contour is formed by an end face formed on the component absorbing the laser radiation, the end face being necessarily bounded by two edges. Advantageously, this end face lies in a plane in which a laser beam, which is scanned over the end face, is focused in order to carry out the process.

Another special feature of laser transmission welding is that, during the process, the two components to be welded are pressed together in a force-fitting manner so that they lie against each other without gaps in the contact area. For this purpose, workpiece holders with clamping tools are used to clamp clamping masks or clamping plungers resting on the transparent component. In order that the necessary mechanical connection of the clamping tool with a clamping mask and/or a clamping plunger does not have a shadowing effect in the imaging beam path, it is known to manufacture this connection from a material that is transparent to the laser radiation and the illumination radiation. A simple solution for this is a glass plate which is placed on the clamping mask and/or the clamping plunger and which is in contact with the clamping tool outside the imaging beam path. Such a glass plate must then be anti-reflective not only for the laser radiation but also for the illumination radiation, which in turn leads to a loss of light for the illumination of the welding contour in addition to the increased manufacturing effort. In the following, the term workpiece holder is to be understood to mean a device which has a receiving area in which the components of an assembly to be welded are connected to one another in a force-fitting manner along the end face in their relative position to one another and to the camera.

Depending on the component geometry, the end face (hereinafter also referred to as welding contour) can be located at different positions on the component and can have a great variety of shapes, sizes and orientations. Often the welding contour corresponds to an annular surface, although the shape of the annular surface can be arbitrary. Additionally or alternatively, the welding contour may be formed by an array of continuous surfaces.

In order to derive a control signal from the image of the welding contour, the welding contour is imaged filling the object field of the camera as much as possible. This means, for example, that an annular welding contour is located in the edge region of the object field. According to the state of the art, illumination is preferably performed with incident light from ring lights or light bars arranged around the component. This may cause shading by the workpiece holder. Often, also due to the scattering of light on the surface of the transparent component, only the surface of the transparent component is imaged and not the end face of the absorbing component, which determines the actual contour for the welding seam to be made.

To dispense with welding controlled by the actual contour and to control the laser according to a pre-programmed target contour leads to a reject rate that is only detected in subsequent process steps, e.g. during a leak test, stress measurement, and/or other subsequent processes/steps that are conceivable for the person skilled in the art.

### SUMMARY OF THE INVENTION

It is the object of the invention to provide a method for creating a high-contrast camera image of the end face of the absorbing component of an assembly to be welded by laser transmission welding.

It is also the object of the invention to provide a device suitable to carry out the method.

The object is achieved by a method for generating a camera image from which a welding contour can be derived along which an assembly is to be welded in an image processing-assisted laser transmission welding process. A workpiece holder with a receiving area in which the assembly is fixed is provided. The assembly comprises a laser radiation absorbing component having an end face bounded by at least one edge formed with at least one wall surface of the absorbing component. Said end face represents the welding contour. A component that is transparent to the laser radiation is arranged on the end face.

Further, a camera having a camera axis is provided, wherein the camera axis is aligned with the assembly.

To capture the camera image, the assembly is illuminated and the camera is triggered. It is essential to the method of the invention that the transparent component is illuminated from a side facing away from the camera.

Advantageously, at least one illumination beam is partially directed onto the wall surface, grazing the edge, whereby the edge is imaged in the camera image as a light-dark transition and the relative position of the welding contour adjacent to the edge is derived from the relative position of the image of the edge in the camera image.

Alternatively, it is also advantageous if at least one illumination beam is coupled into the transparent component in such a way that it is transmitted within the transparent component, whereby radiation components of the illumination beam impinging on the adjacent end face are deflected or absorbed in the direction of the camera, as a result of which the end face is imaged brighter or darker than a background in the camera image.

It is also possible to create a sequence of camera images while the camera is pivoted around a pivot point, thus imaging the welding seam from different directions.

The object is achieved by a device for generating a camera image from which a welding contour can be derived along which an assembly, comprising a component transparent to a laser radiation and a component absorbing the laser radiation, is to be welded in an image processing-assisted laser transmission welding process, wherein the absorbing component has at least one end face which is bounded by at least one edge formed with at least one wall surface of the absorbing component and represents the welding contour, and the transparent component rests on the end face. The device comprises a workpiece holder having a receiving area within which the assembly is fixed, a camera having a camera axis directed toward the receiving area, wherein an object plane of the camera lies within the receiving area in which the welding contour of a received assembly is located, and an illumination unit having at least one light source emitting an illumination beam.

It is essential to the invention that the at least one light source is arranged on or in the workpiece holder and is directed into the receiving area.

It is advantageous if the at least one light source is arranged below the object plane and inclined to the camera axis, whereby the at least one illumination beam directly illuminates at least a portion of the at least one edge in a received assembly in a grazing manner.

Alternatively, the at least one light source is advantageously arranged above the object plane, aligned with, parallel to or inclined to the camera axis, whereby the at least one illumination beam, scattered in a received assembly, indirectly illuminates at least a portion of the at least one edge, grazing the latter.

Depending on the location of the welding seam on the component, it is more convenient either for the at least one light source to be arranged inside the receiving area or for it to be located outside the receiving area.

It can also be advantageous if the at least one light source is aligned above the object plane and parallel to the object plane, whereby the at least one illumination beam is coupled into a received assembly, into the transparent component, and radiation components impinging on the adjacent end face are deflected or absorbed in the direction of the camera.

Advantageously, instead of the at least one light source, there is an opening in the workpiece holder through which the illumination beam is coupled into the workpiece holder.

### BRIEF DESCRIPTION OF THE DRAWINGS

The invention will be explained in more detail below by means of embodiment examples with the aid of drawings, wherein:

FIG. 1a shows a schematic diagram of a first embodiment example of a device in a sectional view;

FIG. 1b is a top view of a tool holder according to the first embodiment example of a device;

FIG. 2 is a schematic diagram of a tool holder according to a second embodiment example of a device in a sectional view;

FIG. 3 is a schematic diagram of a tool holder according to a third embodiment example of a device in a sectional view;

FIG. 4 is a schematic diagram of a tool holder according to a fourth embodiment example of a device in a sectional view;

FIG. 5 is a schematic diagram of a tool holder according to a fifth embodiment example of a device in a sectional view.

### DETAILED DESCRIPTION OF THE DRAWINGS

Using a method according to the invention, at least one camera image is generated from which a welding contour can be derived along which an assembly 1 is to be welded in an image processing-assisted laser transmission welding process.

The method begins with the provision of a workpiece holder 4 with a receiving area 4.1 in which the assembly 1 is fixed. The assembly 1 includes a transparent component 2 and an absorbing component 3. The absorbing component 3 largely absorbs laser radiation suitable for welding the assembly 1 and has at least one end face 3.1 bounded by at least one edge 3.3 formed with a wall surface 3.2 of the absorbing component 3. Said at least one end face 3.1 represents the welding contour. For the sake of simplicity, the following description will assume an end face 3.1, which may also consist of several spaced-apart areas. The transparent component 2 rests on the end face 3.1, forming a boundary surface. It is largely transparent to a laser beam suitable for welding the assembly 1 and is pressed against the end face 3.1 by clamping means to produce a surface contact good for laser transmission welding in a contact area between the end face 3.1 and the surface of the transparent component 2, via the boundary surface.

To weld the assembly 1, a laser beam is passed over the assembly 1 along the end face 3.1, and the transparent component 2 is penetrated by the laser beam, which impinges on the absorbing component 3 and heats it. Due to heat conduction, the laser transparent component 2 is heated in a contact area formed with the absorbing component 3, and fusion and formation of a welding seam occur. The actual welding is not the object of the method described here.

Furthermore, a camera 5 with a camera axis 5.0 is provided for carrying out the method and is directed with the camera axis 5.0 toward the assembly 1 fixed in the workpiece holder 4. The entire assembly 1 does not have to be in the object field of the camera 5 in this case. It is sufficient to have a section containing the end face 3.1 and a reference base on the assembly 1 to be able to derive the position and orientation of the end face 3.1, in addition to the shape and size of the end face 3.1, from the camera image.

A camera image is then generated with the camera 5, with the transparent component 2 being simultaneously illuminated from a side facing away from the camera 5. Any light reflected from the transparent component 2 can therefore not fall into the camera 5. The camera 5 can also be pivoted around a pivot point 5.2 while recording a sequence of camera images.

The illumination leads to a light-dark transition (contrast jump) in the camera image along at least one edge 3.3 of the end face 3.1, and the end face 3.1 can appear brighter or darker compared to a background.

If the end face 3.1 is a closed surface, it has a circumferential wall surface 3.2 and correspondingly a circumferential edge 3.3.

If the end face 3.1 is an annular surface or the portion of an annular surface, it has two circumferential wall surfaces 3.2, representing an inner and an outer wall surface, and correspondingly two edges 3.3, representing an inner and an outer edge in a typically pot-like absorbing component 3.

The embodiment examples described below for the method are more or less advantageously suitable depending on the shape of the assembly 1 and the shape and position of the end face 3.1 determined by it. The embodiment examples described later for a device suitable for carrying out the method are also more or less advantageous depending on the shape of the assembly 1 and the shape and position of the end face 3.1 determined by it.

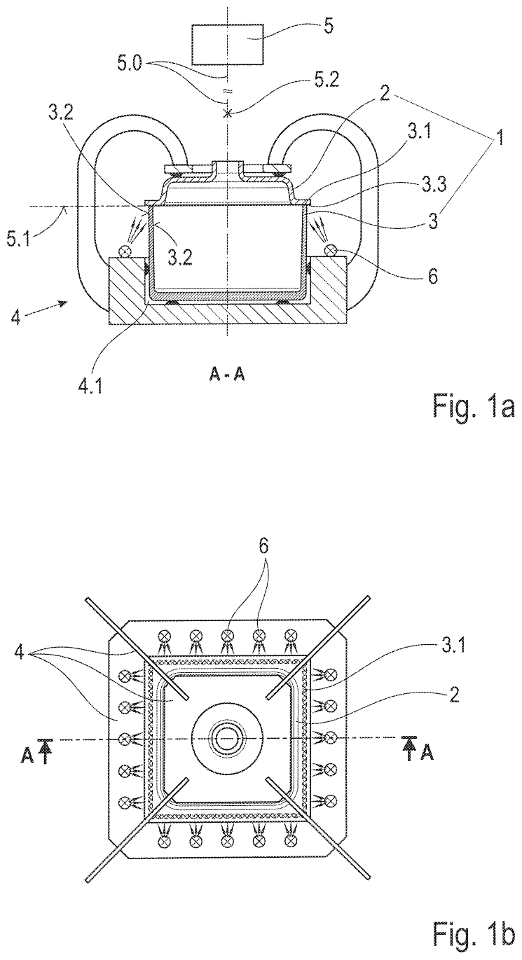

Embodiments of the method and device will be explained below on the basis of an assembly 1 to be welded in the form of a so-called pot-and-lid assembly. This can be, for example, a container with a filler neck, as shown in FIGS. 1-3.

According to a first embodiment example for the method, at least one illumination beam is directed onto the assembly 1 such that it partially impinges on one of the at least one wall surfaces 3.2, grazing one of the at least one edges 3.3. In the camera image, the relevant edge 3.3 is shown as a contrast jump. Knowing the relative position of the image of the relevant edge 3.3 in the camera image, the relative position of the welding contour adjacent to the edge 3.3 can be derived. If the end face 3.1 has two edges 3.3, as shown in FIGS. 1-3, it is sufficient if one of the two edges 3.3 is illuminated according to the invention. Since the relevant edge 3.3 bounds the end face 3.1, the length, shape, position and orientation of the end face 3.1 can be derived from the length, shape, position and orientation of the edge 3.3.

Practically, this type of illumination can be directed at the assembly 1 from outside, as shown in FIGS. 1a-1b, whereby the relevant edge 3.3 is an outer edge and the relevant wall surface 3.2 is then an outer wall surface.

In particular, for assemblies 1 in which the end face 3.1 is adjacent to an outermost peripheral surface of the absorbing component 3, this design of illumination is advantageous. It is irrelevant whether the transparent component 2 projects beyond the outer edge or terminates with it. The illumination beam only has to be directed at the assembly 1 in such a way that no parts of the beam can fall into the camera 5.

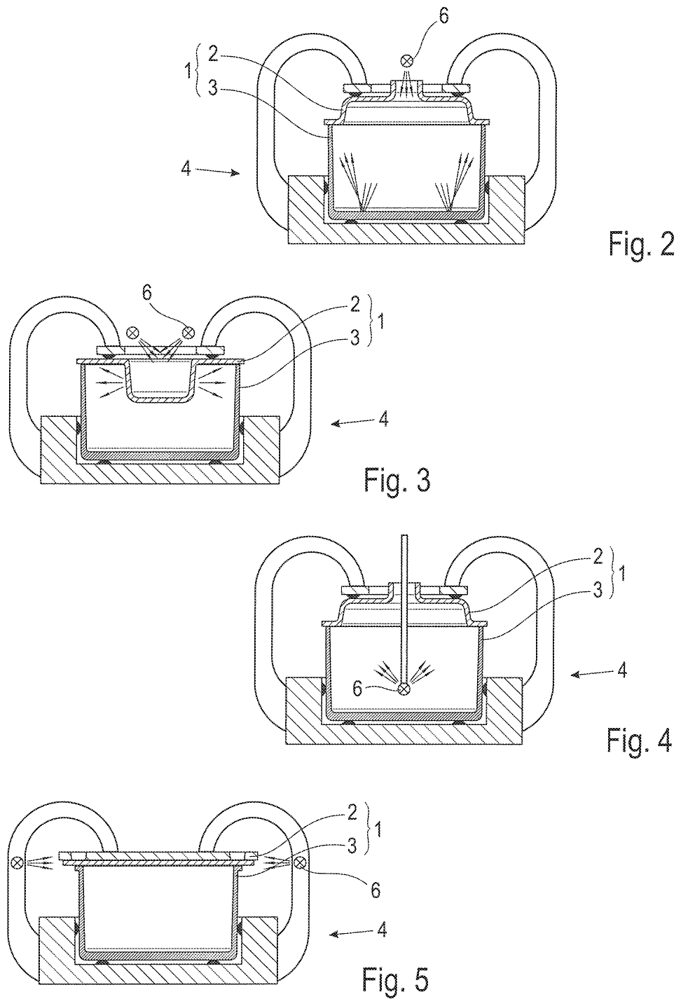

This type of illumination can alternatively be directed into the interior of the assembly 1 or from the interior, in which case the relevant edge 3.3 is an inner edge and the relevant wall surface 3.2 is then an inner wall surface. This design of the illumination is particularly suitable for assemblies 1 which contain a container bounded by the wall surface 3.2 as the absorbing component 3 and an opening is present in the container or, as shown in FIGS. 2-3, in the covering transparent component 2. The inside of the container (cavity) is then illuminated from the inside by coupling and diffusely scattering the at least one illumination beam through the opening, thus illuminating the cavity of the part of the transparent component 2 covering the container. The cavity acts as the actual light source here.

According to a second embodiment example of the method, at least one illumination beam is coupled into the transparent component 2 such that it is propagated within the transparent component 2 so that the latter acts as a secondary light source. In the process, the radiation components of the illumination beam impinging on the boundary surface of the absorbing component 3 formed with the end face 3.1 are deflected or absorbed in the direction of the camera 5, as a result of which the end face 3.1 is imaged brighter or darker in the camera image compared with a background formed by the transparent component 2. The transparent component 2 acts as the actual light source here.

Whether radiation components of the illumination beam are reflected, scattered or absorbed depends in particular on the surface of the boundary surface, but also on the material of the absorbing component 3 and its properties related to the wavelength of the illumination beam. In any case, a contrast jump occurs at the edge 3.3, which can be an inner and/or outer edge.

Such an illumination design is advantageously suitable for assemblies 1 in which the transparent component 2 is formed by a planar plate, as shown in FIG. 5.

In all embodiments, the illumination is advantageously provided by a plurality of illumination beams, each emitted by a light source 6, which are directed onto the assembly 1 depending on the relative position, shape and size of the end face 3.1.

Accordingly, the embodiments of a device suitable for carrying out the method differ.

A device according to the invention basically comprises a workpiece holder 4, with a receiving area 4.1 within which the assembly 1 is fixed, and a camera 5, with a camera axis 5.0 directed toward the receiving area 4.1. In this case, an object plane 5.1 of the camera 5 lies within the receiving area 4.1. The device further includes an illumination unit with at least one light source 6 arranged on or in the workpiece holder 4. The at least one light source 6 emits an illumination beam that is directed into the receiving area 4.1. Depending on the specific embodiment, the light source 6 may be a diffuse-emitting light source 6, a focusing light source 6, or a telecentric-emitting light source 6.

To ensure that the welding contour is optimally imaged in the camera image or can be derived from the camera image, an assembly 1, as already described, is arranged in the receiving area 4.1 in such a way that the end face 3.1 lies in the object plane of the camera 5.1. Deviations from this have no effect on the imaging quality as long as they are within the depth of field range of the camera 5.

In a first embodiment example for the device, shown in FIGS. 1a-1b, the illumination unit contains several light sources 6, each emitting an illumination beam, which are arranged below the object plane 5.1 and inclined to the camera axis 5.0. The individual illumination beams each directly illuminate a portion of the edge 3.3 of a received assembly 1 in a grazing manner. The absorbing component 3 shades the illumination beams to an extent limited by the circumferential edge 3.3, so that a contrast jump occurs in the camera image along the edge 3.3.

Alternatively, the inner edge of the absorbing component 3 can be illuminated as edge 3.3. For this purpose, the illumination unit has at least one light source 6 emitting an illumination beam, which is arranged above the object plane 5.1 in alignment with, parallel to or inclined to the camera axis 5.0, whereby the at least one illumination beam, scattered in a received assembly 1, indirectly illuminates at least a portion of the relevant edge 3.3, grazing the latter.

According to a second embodiment example, shown in FIG. 2, exactly one light source 6 is fixed to the workpiece holder 4, radiating in the direction of the camera axis 5.0 into the receiving area 4.1.

For another embodiment of the transparent component 2, e.g. as shown in FIG. 3, according to a third embodiment example for the device, several light sources 6, forming a ring arrangement, are advantageously provided inclined to the camera axis 5.0 on the workpiece holder 4 above the receiving area 4.1. The light sources 6 arranged outside the receiving area 4.1 preferably emit a telecentric illumination beam in order to couple the light into the cavity of the absorbing component 3, if possible without shading the illumination beam.

The light is scattered within the cavity, illuminating the transparent component 2, bounded by the edge 3.3, which is the inner edge in this case.

According to a fourth embodiment example, shown in FIG. 4, a light source 6, preferably a diffuse-emitting light source, is arranged within the receiving area 4.1. As in the second and third embodiment examples, the edge 3.3, specifically the inner edge, becomes visible here in the camera image due to a contrast jump, with the adjacent end face 3.1 appearing dark.

A fifth embodiment example, shown in FIG. 5, differs from the aforementioned embodiment examples in that the at least one light source 6 of the illumination unit is attached to the workpiece holder 4 in such a way that the emitted telecentric illumination beam is aligned above and parallel to the object plane 5.1, whereby the at least one beam is directly coupled into the transparent component 2. Preferably, there are a plurality of light sources 6 uniformly distributed around the peripheral surface of the transparent component 2. The coupled illumination beams are transmitted in the transparent component 2 by reflection and scattering or coupled out by refraction, so that the transparent component 2 appears self-luminous in the camera image. A boundary transition formed between the boundary surface at the transparent component 2 and the end face 3.1, differs in its effect on the illumination radiation in contrast to a boundary transition between the transparent component 2 adjacent to air. The illumination radiation incident here is comparatively more or less deflected toward the camera 5 or coupled out into the absorbing component 3. More precisely, in the boundary transition, between the end face 3.1 and the transparent component 2, the light impinging on the formed boundary transition behaves differently than in a boundary transition to air. Either more or less light can reach the camera 5 from the boundary area with the end face 3.1 than from the boundary areas with air, so that the end face 3.1 appears brighter or darker in the camera image.

In particular, this embodiment example for a device is also suitable for quality control of a welding seam. The welded areas of the welding seam interact differently with the illumination radiation than any non-welded areas within the welding seam.

All of the above embodiments are modified as further embodiments, not shown in the drawings, in which an opening is provided in the workpiece holder 4 at the respective location of the arrangement of the light sources 6, through which opening the illumination beam of the light source 6 arranged outside the workpiece holder 4 is coupled in. Advantageously, an exit end of an optical fiber is arranged in the respective opening.

### LIST OF REFERENCE NUMERALS

1 assembly
2 transparent component
3 absorbing component
3.1 end face
3.2 wall surface
3.3 edge
4 workpiece holder
4.1 receiving area
5 camera
5.0 camera axis
5.1 object plane
5.2 pivot point
6 light source

1. A method for generating a camera image from which a welding contour can be derived along which an assembly is to be welded in an image processing-assisted laser transmission welding process, comprising the following process steps:
providing a workpiece holder with a receiving area, in which the assembly is fixed, comprising a laser radiation absorbing component having an end face bounded by at least one edge formed with at least one wall surface of the absorbing component and representing the welding contour, and a component which is transparent to the laser radiation and rests on the end face;
providing a camera, with a camera axis, and directing the camera axis toward the assembly;
illuminating the assembly, while a camera image is simultaneously generated with the camera,
wherein the transparent component is illuminated from a side facing away from the camera.

2. The method according to claim 1,
at least one illumination beam is partially directed onto the wall surface, grazing the edge, whereby the edge is imaged in the camera image as a light-dark transition and a relative position of the welding contour adjacent to the edge is derived from a relative position of the image of the edge in the camera image.

3. The method according to claim 1, wherein:
at least one illumination beam is coupled into the transparent component in such a way that the illumination beam is transmitted within the transparent component, whereby radiation components of the illumination beam impinging on the adjacent end face are deflected or absorbed in a direction of the camera, as a result of which the end face is imaged brighter or darker than a background in the camera image.

4. The method according to claim 1, wherein a sequence of camera images is generated while the camera is pivoted around a pivot point.

5. A device for generating a camera image from which a welding contour can be derived along which an assembly, comprising a transparent component transparent to a laser radiation and an absorbing component absorbing the laser radiation, is to be welded in an image processing-assisted laser transmission welding process, wherein the absorbing component has at least one end face which is bounded by at least one edge formed with at least one wall surface of the absorbing component and represents the welding contour, and the transparent component rests on the end face, the device comprising:
a workpiece holder having a receiving area within which the assembly is fixed,
a camera having a camera axis directed toward the receiving area, wherein an object plane of the camera lies within the receiving area in which the welding contour of a received assembly is located, and
an illumination unit having at least one light source emitting an illumination beam, wherein the at least one light source is arranged on or in the workpiece holder and is directed into the receiving area.

6. The device according to claim 5, wherein
at least one light source is arranged below the object plane and inclined to the camera axis, whereby the at least one illumination beam directly illuminates at least a portion of the at least one edge in a received assembly in a grazing manner.

7. The device according to claim 5, wherein
the at least one light source is arranged above the object plane in alignment with, parallel to, or inclined to the camera axis, whereby the at least one illumination beam, scattered in a received assembly, indirectly illuminates at least a portion of the at least one edge, grazing the latter.

8. The device according to claim 6, wherein the at least one light source is arranged within the receiving area.

9. The device according to claim 6, wherein the at least one light source is arranged outside of the receiving area.

10. The device according to claim 5, wherein:
the at least one light source is aligned above the object plane and parallel to the object plane, whereby the at least one illumination beam is coupled into a received assembly, into the transparent component, and radiation components impinging on the adjacent end face are deflected or absorbed in a direction of the camera.

11. The device according to claim 6, wherein, instead of the at least one light source, there is an opening in the workpiece
